(19) United States
(12) Patent Application Publication
(10) Pub. No.: US 2024/0249395 A1
(43) Pub. Date: Jul. 25, 2024

(54) SYSTEMS AND METHODS FOR CONTRAST DOSE REDUCTION

(71) Applicant: Subtle Medical, Inc., Menlo Park, CA (US)

(72) Inventors: Gajanana Keshava DATTA, Sunnyvale, CA (US); Srivathsa PASUMARTHI VENKATA, Sunnyvale, CA (US); Enhao GONG, Sunnyvale, CA (US)

(21) Appl. No.: 18/607,814

(22) Filed: Mar. 18, 2024

Related U.S. Application Data
(63) Continuation of application No. PCT/US2022/044850, filed on Sep. 27, 2022.
(60) Provisional application No. 63/249,974, filed on Sep. 29, 2021.

(51) Int. Cl. G06T 5/60; G01R 33/50; G01R 33/56; G06T 7/00

(52) U.S. Cl. CPC G06T 5/60; G01R 33/50; G01R 33/5608; G06T 7/0002; G06T 2207/10088; G06T 2207/20081; G06T 2207/20084; G06T 2207/30016; G06T 2207/30096

(57) ABSTRACT

A deep learning-based algorithm has been proposed for contrast dose reduction in MRI, using multi-contrast images and an anomaly-aware attention mechanism. The method comprises: obtaining a multi-contrast image of a subject, where the multi-contrast image comprises an image of a first contrast acquired with a reduced dose of contrast agent; generating an anomaly mask using a first deep learning network; and taking the multi-contrast image and the anomaly mask as input to a second deep network model to generate a predicted image with improved quality.

### CROSS-REFERENCE TO RELATED APPLICATION

This application is a continuation of International Application No. PCT/US2022/044850 filed Sep. 27, 2022, which claims priority to U.S. Provisional Application No. 63/249,974 filed on Sep. 29, 2021, the content of which is incorporated herein in its entirety.

### BACKGROUND

Contrast agents such as Gadolinium-based contrast agents (GBCAs) have been used in approximately one third of Magnetic Resonance imaging (MRI) exams worldwide to create indispensable image contrast for a wide range of clinical applications, but pose health risks for patients with renal failure and are known to deposit within the brain and body for patients with normal kidney function. Recently, deep learning (DL) technique has been used to reduce GBCA dose in volumetric contrast-enhanced MRI by synthesizing high quality contrast-enhanced brain images from reduced dose images in diverse clinical settings, but challenges remain due to variability in scanner hardware and clinical protocols within and across sites. In particular, due to the differences in dose injection timing and image acquisition combined with the nature of certain slow-enhancing lesions, the low-dose images (e.g., MR images acquired at reduced or low dose level of contrast agent) can have reduced signal information.

### SUMMARY

A need exists for methods and systems to improve quality of image acquired with reduced dose level of contrast agent such as Gadolinium-Based Contrast Agents (GBCAs). Another need exists for methods and systems capable of classification or anomaly detection based on reduced dose image with improved performance. The present disclosure provides methods and systems for a deep learning (DL) algorithm that uses multi-contrast MRI images to reduce gadolinium dosage in MRI combined with an anomaly-aware attention mechanism through unsupervised anomaly detection (UAD). In some embodiments, in addition to the contrast agent reduction capability, the methods and systems may also be used to identify the slices/regions of anomalies that can facilitate radiologists in further analysis or process such as triaging.

In an aspect, a computer-implemented method is provided for enhancing image quality and anomaly detection. The method comprises: (a) obtaining a multi-contrast image of a subject, where the multi-contrast image comprises an image of a first contrast acquired with a reduced dose of contrast agent; (b) generating an anomaly mask using a first deep learning network; and (c) taking the multi-contrast image and the anomaly mask as input to a second deep network model to generate a predicted image with improved quality.

In a related yet separate aspect, a non-transitory computer-readable storage medium including instructions that, when executed by one or more processors, cause the one or more processors to perform operations. The operations comprise: (a) obtaining a multi-contrast image of a subject, where the multi-contrast image comprises an image of a first contrast acquired with a reduced dose of contrast agent; (b) generating an anomaly mask using a first deep learning network; and (c) taking the multi-contrast image and the anomaly mask as input to a second deep network model to generate a predicted image with improved quality.

In some embodiments, the multi-contrast image is acquired using a magnetic resonance (MR) device. In some embodiments of the method, the first deep learning network is trained using unsupervised anomaly detection scheme. In some cases, the first deep learning network comprises a variational autoencoder (VAE) model trained only on images without anomaly. In some embodiments, the multi-contrast image comprises an image of a second contrast that is processed by the first deep learning network for generating the anomaly mask. In some cases, the image of the first contrast is T1-weighted image and the image of the second contrast is selected from the group consisting of T2-weighted image, fluid attenuated inversion recovery (FLAIR), proton density (PD), and diffusion weighted (DWI).

In some embodiments, the second deep network model comprises multiple branches. In some cases, an input to at least one of the multiple branches comprises the image of the first contrast and an image of a different contrast. In some cases, an input to at least one of the multiple branches comprises the image of the first contrast and the anomaly mask generated in (b). In some cases, an input to each of the multiple branches comprises at least the image of the first contrast. In some cases, the predicted image with improved quality is generated based on multiple predictions generated by the multiple branches.

In some embodiments, the anomaly mask is further utilized as an attention mechanism for training the second deep learning network model. In some embodiments, the method further comprises displaying the predicted image overlaid with the anomaly mask.

In another aspect, a computer-implemented method is provided for enhancing image quality with anomaly-aware mechanism. The method comprises: obtaining a multi-contrast image of a subject, where the multi-contrast image comprises an image of a first contrast acquired with a reduced dose of contrast agent; providing a deep learning network model comprising a multi-contrast branched architecture; and taking the multi-contrast image and an anomaly mask as input to the deep network model to generate a predicted image with improved quality.

In some embodiments, the multi-contrast branched architecture comprises a first branch configured to process the image of the first contrast and an image of a second contrast. In some cases, the multi-contrast branched architecture comprises a second branch to process the image of the first contrast and the anomaly mask. In some embodiments, the multi-contrast branched architecture comprises at least three branches.

In some embodiments, the anomaly mask is further utilized as an attention mechanism for training the deep learning network model. In some embodiments, the predicted image with improved quality is generated based on multiple predictions generated by the at least three branches. In some cases, the predicted image with improved quality is generated based on an average of the multiple predictions. In some instances, the multi-contrast branched architecture further comprises a deep learning model to take the average of the multiple predictions along with the image of the first contrast as input and output the predicted image with improved quality.

In some embodiments, the multi-contrast image is acquired using a magnetic resonance (MR) device. In some cases, the image of the first contrast is T1-weighted image and the image of the second contrast is selected from the group consisting of T2-weighted image, fluid attenuated inversion recovery (FLAIR), proton density (PD), and diffusion weighted (DWI).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of the training scheme of the Variational Autoencoder (VAE) model to generate a reconstruction.

FIG. 2 shows an example of a workflow of an anomaly mask generation.

FIG. 3 shows an example of multi-contrast branched architecture for synthesizing post-contrast images from low-dose images.

FIG. 4 shows an example of a U-Net style encoder-decoder network architecture.

FIG. 5 schematic shows a method of utilizing the UAD for automated reporting and triaging.

FIG. 6 shows an example of quantitative and qualitative results of the proposed multi-contrast model with UAD-enabled attention mechanism.

FIG. 7 schematically illustrates a platform or system implementing the methods consistent with those described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Gadolinium-based contrast agents (GBCAs) are widely used in magnetic resonance imaging (MRI) exams and have been indispensable for monitoring treatment and investigating pathology in myriad applications including angiography, multiple sclerosis and tumor detection. Recently, the identification of prolonged gadolinium deposition within the tissue, organ and/or body has raised safety concerns about the usage of GBCAs. Reducing the GBCA dose reduces the degree of deposition, but also degrades contrast enhancement and tumor conspicuity. A reduced dose exam that retains contrast enhancement is therefore greatly relevant for patients who need repeated contrast administration (e.g., multiple sclerosis patients) and are at high risk of gadolinium deposition (e.g., children).

Though MRI, Gadolinium-based contrast agents, MRI data and brain examples are primarily provided herein, it should be understood that the present approach can be used in other imaging modality contexts, imaging various other tissues, organs, and/or other contrast-enhanced imaging. For instance, the presently described approach may be employed on data acquired by other types of tomographic scanners including, but not limited to, computed tomography (CT), single photon emission computed tomography (SPECT) scanners, Positron Emission Tomography (PET), functional magnetic resonance imaging (fMRI), or various other types of imaging scanners or techniques wherein a contrast agent may be utilized for enhancing the contrast.

The methods and systems herein provide a deep learning (DL) based algorithm for contrast dose reduction in MRI, using multi-contrast images and an anomaly-aware attention mechanism. The term “multi-contrast” as utilized herein may generally refer to multiple MR imaging sequences such as T1-weighted (e.g., short TE and TR times; contrast and brightness of the image are predominately determined by T1 properties of tissue), T2-weighted (e.g., longer TE and TR times; contrast and brightness are predominately determined by the T2 properties of tissue), fluid attenuated inversion recovery (FLAIR) (e.g., TE and TR times are very long; abnormalities remain bright but normal CSF fluid is attenuated and made dark), proton density (PD), diffusion weighted (DWI) and various other contrasts. Multi-contrast MRI may produce multi-contrast images under different settings but with the same anatomical structure. For example, T1 and T2 weighted images (T1WI and T2WI), as well as proton density and fat-suppressed proton density weighted images (PDWI and FS-PDWI), which can provide complementary information to each other. For instance, T1WI describe morphological and structural information, while T2WI describe edema and inflammation. Further, PDWI provide information on structures such as articular cartilage, and have a high signal-to-noise ratio (SNR) for tissues with little difference in PDWI, while FS-PDWI can inhibit fat signals and highlight the contrast of tissue structures such as cartilage ligaments. In clinical settings, T1WI have shorter repetition time (TR) and echo time (TE) than T2WI, while PDWI usually take shorter than FS-PDWI in the scanning process.

In some cases, the MRI may be utilized to image a subject, a body, or a part of body such as brain. The DL algorithm and methods herein can also be used in speeding up the process of reporting and triaging. The algorithm herein may provide multiple advantages. For example, the proposed multi-contrast branched model with anomaly-aware attention mechanism, has a better quantitative and qualitative performance over the T1-only synthesis DL model. Additionally, the provided deep network architecture may beneficially reduce false negatives in the model relying on a single imaging sequence (e.g., T1-only model), as other contrasts or sequences such as T2 and FLAIR images can offer complimentary information. The methods herein may leverage the complementary information from different contrasts to generate an anomaly mask as well as enhancing the quality of a low-dose image.

In some embodiments, the generated anomaly masks overlaid on the synthesized images (e.g., synthesized contrast-enhanced T1-weighted images (T1+C) images) may provide improved visual guidance that can be conveniently used for radiologist reporting and triaging and various other analysis or applications. The term “contrast-enhanced T1-weighted image” as utilized herein refer to T1-weighted imaging with infusing of contrast enhancement agent Gadolinium (Gad). In some cases, contrast-enhanced MRI may refer to administering contrast media/agents and post-contrast images (e.g., post-contrast T1) may refer to the images obtained at a time after administering a contrast agent (e.g., T1 obtained after administration of intravenous Gadolinium).

In some embodiments, methods and systems herein may provide a multi-contrast deep learning framework for generating a synthesized image with improved quality compared to the input image. For instance, the input image may be an MR image acquired with reduced or lower contrast dose level and the synthesized image may have an improved quality that is similar to the quality of an MR image acquired with full dose of contrast agent (also referred to as full-dose image or post-contrast image). The multi-contrast deep learning framework may comprise an anomaly-aware attention mechanism to focus more on the region where anomaly is detected. The anomaly-aware attention mechanism may be based on an anomaly mask that is generated using an unsupervised anomaly detection scheme.

### Unsupervised Anomaly Detection (UAD) Scheme

Conventional supervised learning for anatomy detection may have drawbacks. For instance, the conventional supervised training requires large and diverse annotated datasets, which are scarce and costly to obtain, and the resulting models are limited to the discovery of lesions which are similar to those in the training data. The methods and systems herein may provide anomaly detection scheme employing unsupervised anomaly detection (UAD) scheme with a variational autoencoder (VAE) model that is trained only on healthy images, for self-reconstruction. In some cases, healthy images may refer to the subject, body part or tissues, etc. captured in the image are healthy or without anomaly. The method may comprise modeling of healthy anatomy with unsupervised deep (generative) representation learning. After the variational autoencoder (VAE) model is trained, it may be used to segment anomalous input images. An autoencoder of the model may construct a healthy version of the input image. The residual between the input image and the autoencoder output, along with some post-processing, helps localize the anomalous regions.

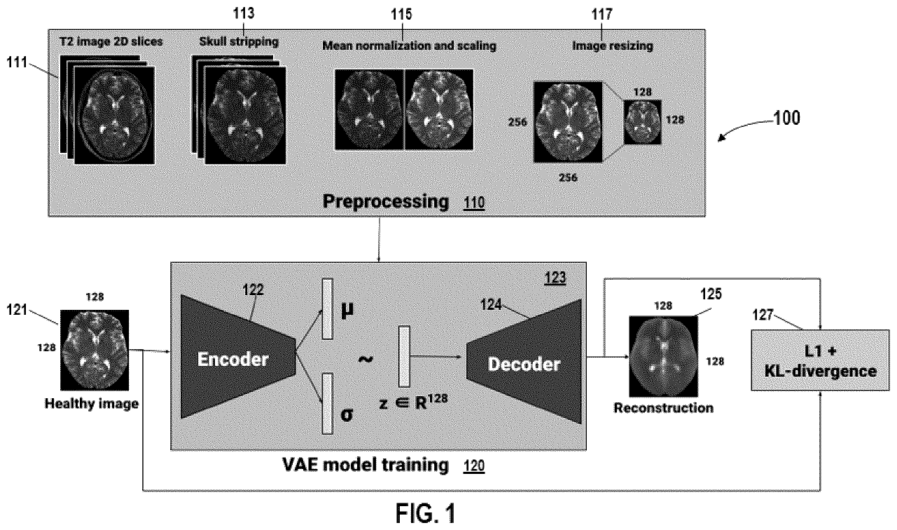

FIG. 1 shows an example of the training scheme 120 of the Variational Autoencoder (VAE) model 123 to generate a reconstruction 125. The model 123 may be trained on healthy images only 121. In some embodiments, the model 123 may be trained to identify an anomaly region or generate an anomaly mask. Based on the specific tissues being examined or the use application, the input image to the anomaly detection model may be a type of contrast (e.g., T2 weighted, FLAIR, etc.) that can provide complementary information that was missing in the low-dose contrast images (e.g., T1 weighted image acquired with reduced dose of contrast agent). For example, due to the differences in dose injection timing and image acquisition combined with the nature of certain slow-enhancing lesions, the low-dose T1 images may miss signal information. The multi-contrast MRI images such as T2 and FLAIR, which are part of the routine protocol of multi-contrast MRI acquisition, can provide complimentary information that is used to recover the contrast signal information. For example, the multi-contrast images such as T2 and FLAIR may have hyper-intensities in and around the regions of lesions and tumors, which can be utilized for anomaly detection.

In the illustrated example, the VAE model 123 may be trained only on healthy T2 images or healthy FLAIR images. In some cases, the training data 121 may comprise only healthy MR images such as volumetric image data (e.g., stack of 2D slices). For example, the training data may include a plurality of 2D image slices capturing a healthy tissue (e.g., brain) that are acquired without contrast agent (e.g., pre-contrast image slice) and/or with reduced contrast dose (e.g., low-dose image slice). The training image data may be acquired in a scanning plane (e.g., axial) or along a scanning orientation. A number of the image slices may be stacked to form a 2.5D volumetric input image. In the illustrated example, the MR images may be brain MR images at a slice resolution of 128×128. The training image data may be acquired at a selected view (e.g., axial, coronal, sagittal, etc.) or with an imaging sequence (e.g., T2, FLAIR, etc.).

In some cases, when the network operates on 2D slices as higher resolution images (e.g., 128×128) it tends to reconstruct finer details which may not be preferred in the subsequent anomaly detection task. The encoder network 122 of the model may be trained to project a healthy input sample to a lower dimensional manifold z, from which a decoder 124 may then try to reconstruct the input. The VAE model Variational may constrain the latent space by leveraging the encoder and decoder networks to parameterize a latent distribution q(z)˜N (zμ, zσ). The VAE may project input data onto a learned mean μ and variance σ, from which a sample is drawn and then reconstructed. The VAE may try to match q(z) to a prior p(z) (typically a multivariate normal distribution) by minimizing the KL-Divergence. For instance, the training framework may weight the reconstruction loss (e.g., L1 loss) against the distribution-matching KL Divergence 127.

In some embodiments, the network may be an encoder-decoder network or a U-net encoder-decoder network. A U-net is an auto-encoder in which the outputs from the encoder-half of the network are concatenated with the mirrored counterparts in the decoder-half of the network. The U-net may replace pooling operations by upsampling operators thereby increasing the resolution of the output. In an example, the encoder network 122 may consist of a series of convolutions (e.g., 5×5 convolutions) with stride 2 and LeakyReLU activation. In an example, the bottleneck layer is a dense layer (e.g., a dimension of 128). In some cases, the decoder 124 may have a series of transpose convolutions (e.g., 5×5 transpose convolutions).

For detecting anomalies in different MR contrast images such as T2 and FLAIR, separate VAE models may be trained on T2 and FLAIR images respectively. Similarly, any other imaging sequence that contains complementary information (e.g., proton density (PD), diffusion weighted (DWI), etc.) may also be used for training the model. In some cases, the training dataset for the VAE models may be obtained without administering contrast agent. Alternatively, based on the use application or tissues being examined, the images processed by the VAE model for generating the anomaly may be T1 images and may be acquired with low-dose of contrast agent.

In some cases, the input datasets may be pre-processed prior to training or inference. In some embodiments, prior to training, the images may be preprocessed 110 as shown in FIG. 1. The input data may include the raw pre-contrast, or low-dose images. As described above, for training the model, the training data may not require labeled data such as anomalies. For example, the training data may comprise pre-contrast (MR images acquired without contrast agent) or low-dose (MR images acquired with lower dose level) MR images capturing healthy tissue. The raw image data may be received from a standard clinical workflow, as a DICOM-based software application or other imaging software applications. Any suitable preprocessing method may be employed to process the training data 111. For example, skull stripping 113, mean normalization and scaling 115, and/or image resizing 117 may be applied to the input image 111 (e.g., T2 mages 2D slices). The skull-stripping operation 113 may be performed to isolate the brain image from cranial or non-brain tissues by eliminating signals from extra-cranial and non-brain tissues using the DL-based library. Based on the tissues, organs and use application, other suitable preprocessing algorithms may be adopted to improve the processing speed and accuracy of diagnosis. The preprocessed images 121 may then be utilized for training the model as described above. For instance, the model may be trained using a combination of L1 reconstruction loss and weighted KL divergence loss, to project the input data onto a learned mean and variance, from which a sample is drawn and reconstructed.

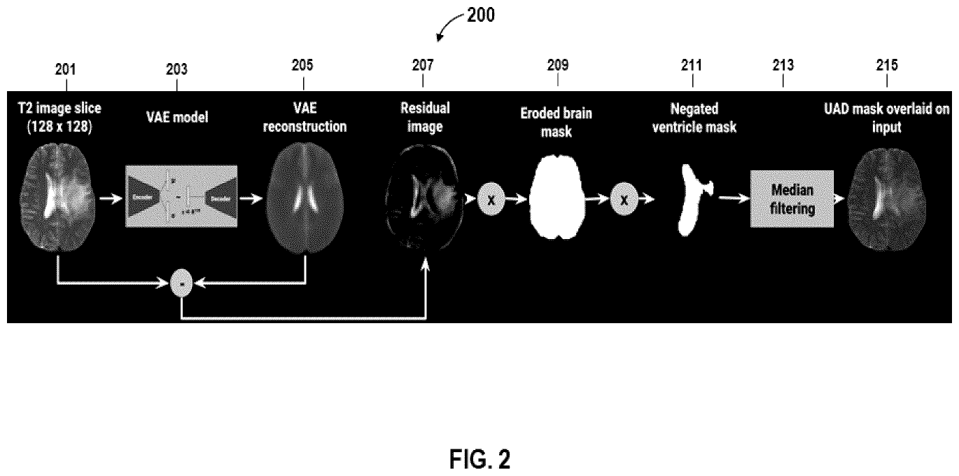

The trained VAE models may then be used for anomaly detection and segmentation. During the anomaly detection stage, the anomalous image may be reconstructed using the trained VAE model and post-processed to generate a UAD mask (anomaly segmentation). FIG. 2 shows an example of a workflow 200 of the UAD mask generation. The illustrated workflow may be UAD mask generated based on T2 images 201. A similar workflow may be applied for FLAIR images, except for the ventricle mask.

During the anomaly detection stage, the anomalous image may be reconstructed using the trained VAE 203 and post-processed as shown in FIG. 2. The detection method may be reconstruction-based method. For example, residual image 207 (e.g., pixel-wise residuals) between the input T2 image slice 201 and the reconstructed image 205 (predicted by the VAE model 203 based on the input T2 image 201) may be used for generating the anomaly mask. The residual image (i.e., difference between the reconstructed image 205 and the input image 201) may contain information about anomalous structures because the anomalous structures, which have never been seen during training, cannot be properly reconstructed from the distribution encoded in the latent space, such that reconstruction errors will be high for the anomalous structures. For example, the VAE model 203 may have high reconstruction loss samples with lesions or tumors, that may not be part of the encoded latent distribution. This makes the pixel-wise residuals 207 i.e., the absolute difference between the input 201 and the VAE reconstruction 205, to contain the anomalous regions.

The post-processing may comprise thresholding the residual image. In some cases, the residual image 207 may be thresholded. The threshold may be selected as for example, a prior quantile of pixel values. In some cases, the post-processing may further comprise applying an eroded brain mask 209 to the residual image 207. The eroded brain mask 209 may help to remove sharp hyperintense regions at the brain-mask boundaries. In some cases, the post-processing may include additional operations based on the contrast or imaging sequence of the MR image. For example, if the input is T2 image, as the ventricle regions are hyperintense in T2 images (ventricle appears very bright in T2 weighted images), these ventricle regions may be removed (negating the ventricle mask 211) such as using the VentMapp3r tool, to obtain the anomaly mask. If the input images are FLAIR images, the operation of negating ventricle mask may not be required in the post-processing. In some cases, the ventricle signals may be computed from the T1-pre contrast (T1 weighted without contrast agent) and then applied on the residual image since the T1 and T2 images are co-registered.

In some cases, based on the type of sequence or contrast images, the post processing follow may comprise more, less or different operations. The post processing follow may comprise any suitable method such as median filter 213 or resizing the anomaly mask to match the size of the original input image. For example, the anomaly mask may be resized to the dimensions of the input image to obtain the final UAD mask. The illustrated example shows a final UAD mask overlaid on the input 215.

Multi-Contrast Branched Model with Attention Mechanism

In some embodiments of the provided framework, it comprises a multi-contrast branched architecture for synthesizing post-contrast images from pre-contrast or low-dose T1 images. The multi-contrast branched architecture may comprise a plurality of separate encoding pathways each corresponding to an input image having a different combination of contrasts. Having separate encoding pathways for the individual contrasts, instead of squashing the multiple contrasts or modalities as channels, performed better, as the separate encoders are able to learn the unique features offered by the different contrasts.

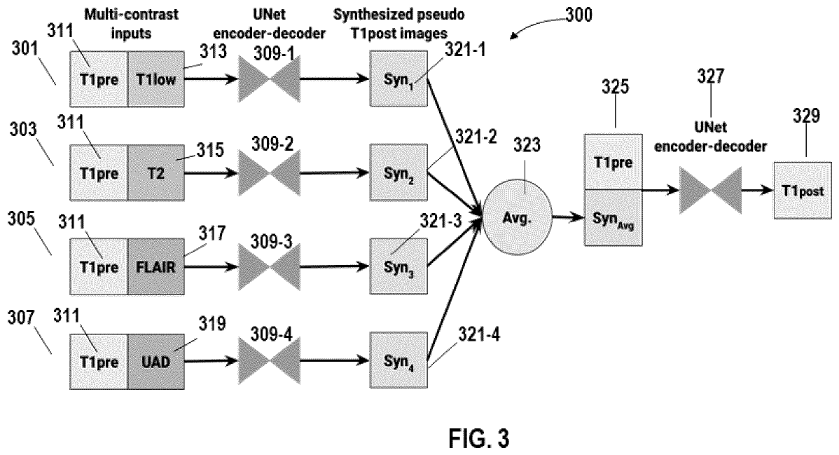

FIG. 3 shows an example of multi-contrast branched architecture 300 for predicting an image with enhanced quality. In some cases, the output image may be a synthesized image having quality same as an image acquired with full-dose of contrast agent. For instance, the input to the network 300 may comprise pre-contrast or low-dose MR images and the output of the network may be a synthesized image having a quality of post-contrast MR image. In some cases, low-dose or reduced dose level may refer to a dose level such as no more than 1%, 5%, 10%, 15%, 20%, 30%, any number higher than 30% or lower than 1%, or any number in-between. In some cases, after deploying the model to a clinical site, a user (e.g., physician) may be permitted to choose a reduced dose level that can be any level in the range from 0 to 30% for acquiring the medical image data. It should be noted that depending on the practical implementation and user desired dose reduction level, the reduced dose level can be any number in a range greater than 30%. The term pre-contrast image may refer to an image acquired with zero contrast dose.

As shown in the example, the model 300 may be trained to synthesize post-contrast images from low-dose T1 images 311. The synthesized post-contrast image may have an improved quality. For example, the improved quality may be same as the quality of an image acquired with full-dose of contrast agent. For instance, the input image may comprise pre-contrast T1 311 and low-dose T1 images 313. As an example, two 3D T₁-weighted images may be obtained for a subject: pre-contrast T1 311 and post-10% dose contrast (e.g., 0.01 mmol/kg) 313.

The multi-contrast branched architecture 300 may comprise a plurality of separate encoding pathways 301, 303, 305, 307. The multi-contrast branched architecture may comprise at least, two, three, four or more branches. Each encoding pathway may correspond to an input image having a different combination of contrasts. For example, an input to at least one of the multiple branches comprises a combination of an image of a first contrast (e.g., T1-weighted) and an image of a different contrast (e.g., T1-weighted, FLAIR, etc.). In some cases, the input to each of the multiple branches may comprise an image of a first contrast acquired with reduced dose of contrast agent. Having separate pathways for the individual contrasts encodings (e.g., T1, T2, FLAIR), instead of squashing the multiple modalities as channels, performed better, as the individual encoder 309-1, 309-2, 309-3, 309-4 of each pathway may be able to learn the unique features offered by the different contrasts. The different input images for each branch may be images acquired using different pulse sequences (e.g., contrast-weighted images such as T1-weighted (T1), T2-weighted (T2), proton density (PD) or Fluid Attenuation by Inversion Recovery (FLAIR), etc.). In some cases, the input to each of the plurality of encoding pathways 301, 303, 305, 307 may comprise at least pre-contrast T1 image 311.

The input data for an individual pathway may comprise two or more different contrasts. For example, the input to a first encoding pathway 301 may comprise a combination of pre-contrast T1 image 311 and a low-dose T1 image 313. The pair of images may be co-registered and processed by the corresponding contrast enhancement model 309-1 to predict a synthesized post-contrast T1 image 321-1. In some cases, the input to a second encoding pathway 302 may comprise a combination of pre-contrast T1 image 311 and a T2-weighted image 315. The combination of different sequences may beneficially leverage other imaging sequence that contains complementary information. The pair of images 311, 315 may be co-registered and processed by the corresponding contrast enhancement model 309-2 to predict a synthesized post-contrast T1 image 321-2. In some cases, the input to a third encoding pathway 303 may comprise a combination of pre-contrast T1 image 311 and a FLAIR image 317. The pair of images 311, 317 may be co-registered and processed by the corresponding contrast enhancement model 309-3 to predict a synthesized post-contrast T1 image 321-3. In some cases, the input to a fourth encoding pathway 304 may comprise a combination of pre-contrast T1 image 311 and the corresponding UAD mask 319. The UAD mask may be generated using the method as described above. For example, the UAD mask may be generated using the T2 or FLAIR images. The input of the pre-contrast T1 image 311 and UAD 319 may be co-registered and processed by the corresponding contrast enhancement model 309-4 to predict a synthesized post-contrast T1 image 321-4.

In some cases, the UAD mask 319 may be utilized both as input and as an attention mechanism by weighting the loss function (e.g., L1-loss). For example, the L1-loss may be weighted with the UAD mask to make the model pay more attention to the anomalous regions. The anomaly-aware attention mechanism may be achieved through adding the UAD masks as part of the input and also weighting the L1-loss.

In some embodiments, each individual branch may comprise a contrast enhancement model 309-1, 309-2, 309-3, 309-4 to process different combinations of input images. In some embodiments, the individual branches 301, 303, 305, 307 may be pre-trained with a post-contrast image as the target. The contrast enhancement model 309-1, 309-2, 309-3, 309-4 may be pre-trained with a full-dose contrast image as target along with the respective contrast images (e.g., T1 pre-contrast, T1 low-dose image, T2 low dose, T2, FLAIR, etc.). In some cases, the deep learning model (e.g., contrast enhancement model) may be trained with volumetric images (e.g., augmented 2.5D images) acquired from multiple orientations (e.g., three principal axes). The contrast enhancement model 309-1, 309-2, 309-3, 309-4 may be a trained deep learning model for enhancing the quality of volumetric MRI images (such that the appearance of the image mimics a full-dose MR image). In some embodiments, the model may include an artificial neural network that can employ any type of neural network model, such as a feedforward neural network, radial basis function network, recurrent neural network, convolutional neural network, deep residual learning network and the like. In some embodiments, the machine learning algorithm may comprise a deep learning algorithm such as convolutional neural network (CNN). Examples of machine learning algorithms may include a support vector machine (SVM), a naïve Bayes classification, a random forest, a deep learning model such as neural network, or other supervised learning algorithm or unsupervised learning algorithm. The model network may be a deep learning network such as CNN that may comprise multiple layers. For example, the CNN model may comprise at least an input layer, a number of hidden layers and an output layer. A CNN model may comprise any total number of layers, and any number of hidden layers. The simplest architecture of a neural network starts with an input layer followed by a sequence of intermediate or hidden layers, and ends with output layer. The hidden or intermediate layers may act as learnable feature extractors, while the output layer in this example provides 2.5D volumetric images with enhanced quality (e.g., enhanced contrast). Each layer of the neural network may comprise a number of neurons (or nodes). A neuron receives input that comes either directly from the input data (e.g., low quality image data, image data acquired with reduced contrast dose, etc.) or the output of other neurons, and performs a specific operation, e.g., summation. In some cases, a connection from an input to a neuron is associated with a weight (or weighting factor). In some cases, the neuron may sum up the products of all pairs of inputs and their associated weights. In some cases, the weighted sum is offset with a bias. In some cases, the output of a neuron may be gated using a threshold or activation function. The activation function may be linear or non-linear. The activation function may be, for example, a rectified linear unit (ReLU) activation function or other functions such as saturating hyperbolic tangent, identity, binary step, logistic, arcTan, softsign, parameteric rectified linear unit, exponential linear unit, softPlus, bent identity, softExponential, Sinusoid, Sinc, Gaussian, sigmoid functions, or any combination thereof.

In some embodiments, the contrast enhancement model 309-1, 309-2, 309-3, 309-4 may be an encoder-decoder network or a U-net encoder-decoder network. A U-net is an auto-encoder in which the outputs from the encoder-half of the network are concatenated with the mirrored counterparts in the decoder-half of the network. The U-net may replace pooling operations by upsampling operators thereby increasing the resolution of the output.

In some embodiments, the contrast enhancement model 309-1, 309-2, 309-3, 309-4 for enhancing the volumetric image quality or synthesizing full-dose image may be trained using supervised learning. For example, to train the deep learning network, pairs of pre-contrast and low-dose images as input and the full-dose image as the ground truth from multiple subjects, scanners, clinical sites or databases may be provided as training dataset. For instance, three scans including a first scan with zero contrast dose, a second scan with a reduced dose level and a third scan with full dose may be operated. The reduced dose image data used for training the model, however, can include images acquired at various reduced dose level such as no more than 1%, 5%, 10%, 15%, 20%, 30%, any number higher than 30% or lower than 1%, or any number in-between. For example, the input data may include image data acquired from two scans including a full dose scan as ground truth data and a paired scan at a reduced level (e.g., zero dose or any level as described above). Alternatively, the input data may be acquired using more than three scans with multiple scans at different levels of contrast dose. In some cases, low dose may refer to no more than 10% of the original standard of care dose of the Gad agent. In some cases, low dose may be any number between 10-50% of the original standard of care dose of the Gad agent. In some cases, the T2 and FLAIR may be obtained before administering the contrast dose.

Additionally, the input data may comprise augmented datasets obtained from simulation. For instance, image data from clinical database may be used to generate low quality image data mimicking the image data acquired with reduced contrast dose. In an example, artifacts may be added to raw image data to mimic image data reconstructed from images acquired with reduced contrast dose.

In some embodiments, the individual encoder-decoder pathways may take the combination of the T1 pre-contrast with the respective contrasts (e.g., T1 low-dose, T2, FLAIR or the UAD masks) and output respective pseudo full-dose images 321-1, 321-2, 321-3, 321-4. The pseudo full-dose images 321-1, 321-2, 321-3, 321-4 may be combined such as averaged and further combined with the original T1 pre-contrast image to form an input 325 to a final encoding pathway 327. The combination of the T1 pre-contrast image and the averaged results from the plurality of encoding pathways may be processed by a final encoding pathway 327 to output the final output image 329.

The learned contrast enhancement signals from the individual pathways may be boosted in the final encoder-decoder pathway to produce the final post-contrast image. The separate encoding pathways may be separately pre-trained with the respective combinations, to predict the full-dose images.

In some embodiments, the multi-contrast model may be trained with a combination of L1, SSIM and perceptual losses. The L1-loss may be weighted with the UAD mask to make the model pay more attention to the anomalous regions. The anomaly-aware attention mechanism may be achieved through adding the UAD masks as part of the input and also weighting the L1-loss. In some cases, a perceptual loss from a convolutional network (e.g., VGG-19 network consisting of 19 layers including 6 convolution layers, 3 Fully connected layer, 5 MaxPool layers and 1 SoftMax layer which is pre-trained on ImageNet dataset) may be employed. The perceptual loss is effective in style-transfer and super-resolution tasks. For example, the perceptual loss can be computed from the third convolution layer of the third block (e.g., block3 conv3) of a VGG-19 network, by taking the mean squared error (MSE) of the layer activations on the ground truth and prediction.

An example of the loss function between ground-truth full-dose I_{T1ce} (contrast-enhanced T1) and deep learning synthesized full-dose I*_{T1ce} (synthesized contrast-enhanced T1) is the following:

$$L ( {I}_{T 1 ce ,} {I}_{T 1 ce}^{*} = {λ}_{L 1} {L}_{L 1} ( {M}_{UAD} {I}_{T 2} ) + {λ}_{SSIM} {L}_{SSIM} + {λ}_{VGG} {L}_{VGG}$$

where λs are the respective loss weights and M_{UAD} is the UAD mask computed on the T2 image. The λ values may be empirically chosen or may be determined based on the results on the validation set.

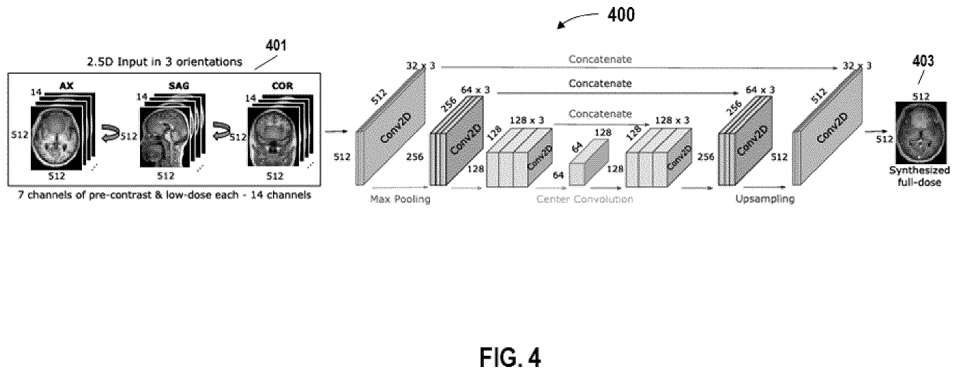

FIG. 4 shows an example of a U-Net style encoder-decoder network architecture 400, in accordance with some embodiments herein. In the illustrated example, each encoder block has three 2D convolution layers (3×3) with ReLU followed by a maxpool (2×2) to downsample the feature space by a factor of two. The decoder blocks have a similar structure with maxpool replaced with upsample layers. To restore spatial information lost during downsampling and prevent resolution loss, decoder layers are concatenated with features of the corresponding encoder layer using skip connections. The network may be trained with a combination of L1 (mean absolute error) and structural similarity index (SSIM) losses. Such U-Net style encoder-decoder network architecture may be capable of producing a linear 10× scaling of the contrast uptake between low-dose and zero-dose, without picking up noise along with the enhancement signal.

In some cases, as shown in FIG. 4, the input data to the network may include a plurality of augmented volumetric images. For example, the input 2.5D volumetric image may be reformatted into multiple axes such as principal axes (e.g., sagittal, coronal, and axial) to generate multiple reformatted volumetric images (e.g., SAG, AX, COR). It should be noted that the 2.5D volumetric image can be reformatted into any orientations that may or may not be aligned with the principal axes. In the example, seven slices each of pre-contrast and low-dose images are stacked channel-wise to create a 14-channel input volumetric data for training the model to predict the central full-dose slices 403.

### Automated Report of Anomalous Slices for Triaging

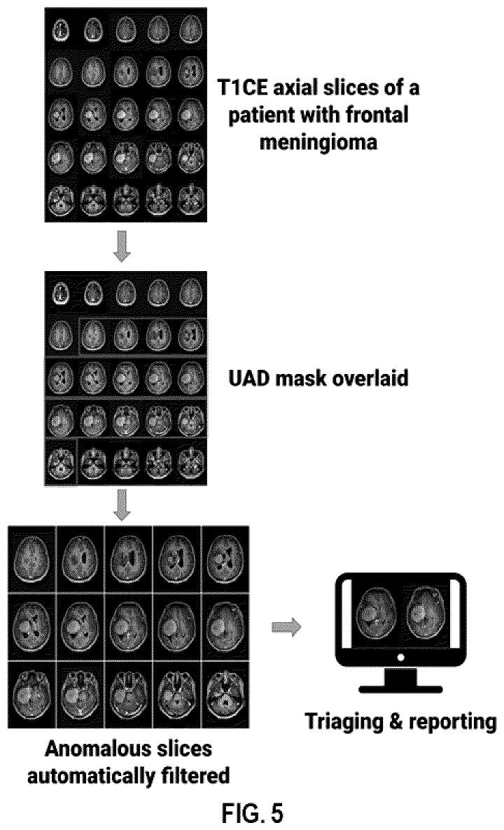

In some embodiments, the anomaly masks (UAD mask) generated using the T2 or FLAIR images may be overlaid on the synthesized T1 post-contrast images after image registration. Systems and methods herein may provide an improved visualization tool for a user to better visualize the anomaly. FIG. 5 schematic shows a method of utilizing the UAD for automated reporting and triaging. In FIG. 5, an anomaly mask (UAD mask) is generated using the method herein, and is overlaid on the synthesized image (e.g., synthesized contrast-enhanced T1-weighted images (T1+C) images). The UAD mask overlaid to the T1+C image may provide improved visual guidance that can be conveniently used for radiologist reporting and triaging. In some cases, one or more anomalous slices may be automatically filtered or identified from the stack of slices which may be used as a means of facilitating radiologist report writing and triaging. For example, a report may be automatically generated based on the identified one or more anomalous slices.

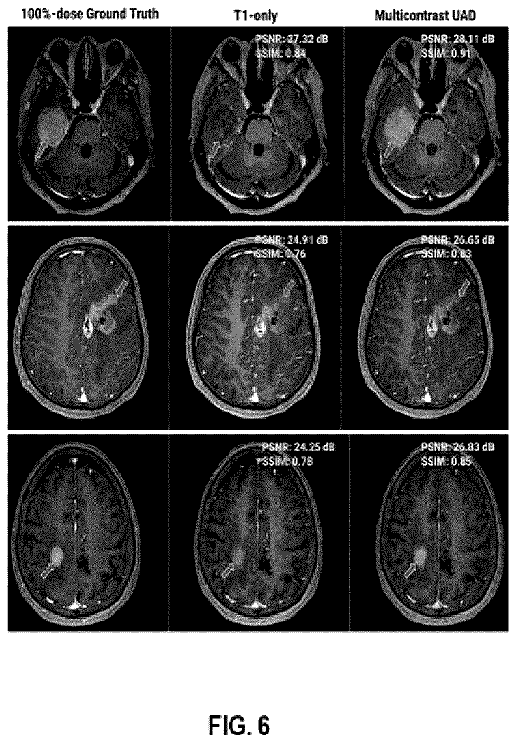

FIG. 6 shows an example of quantitative and qualitative results of the proposed multi-contrast model with UAD-enabled attention mechanism. The results show that the proposed model performs better than the T1-only model. In some cases, the contrast to overlay the anomaly masks may be T1 images to show the enhancing lesions. However, other contrasts can be overlaid on any of the registered images such as T2, FLAIR, PD, DWI, etc, depending on the tissues being imaged or user preference. In some cases, a user may be permitted to select the output image format such as defining which image (e.g., T1, T2, FLAIR, PD, DWI) is utilized to display the overlaying UAD mask with.

### System Overview

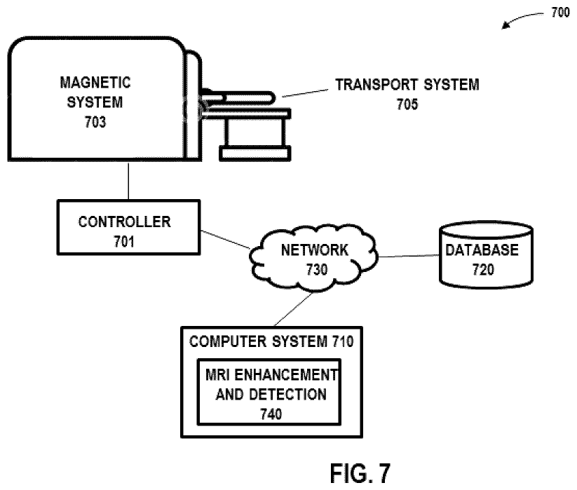

The systems and methods can be implemented on existing imaging systems such as but not limited to MR imaging systems without a need of a change of hardware infrastructure. Alternatively, the systems and methods can be implemented by any computing systems that may not be coupled to the MR imaging system. For instance, methods and systems herein may be implemented in a remote system, one or more computer servers, which can enable distributed computing, such as cloud computing. FIG. 7 schematically illustrates an example MR system 700 comprising a computer system 710 and one or more databases operably coupled to a controller over the network 730. The computer system 710 may be used for further implementing the methods and systems as described for processing the medical images (MR images) for contrast dose reduction or image enhancement.

The controller 701 may be operated to provide the MRI sequence controller information about a pulse sequence and/or to manage the operations of the entire system, according to installed software programs. The controller may also serve as an element for instructing a patient to perform tasks, such as, for example, a breath hold by a voice message produced using an automatic voice synthesis technique. The controller may receive commands from an operator which indicate the scan sequence to be performed. The controller may comprise various components such as a pulse generator module which is configured to operate the system components to carry out the desired scan sequence, producing data that indicate the timing, strength and shape of the RF pulses to be produced, and the timing of and length of the data acquisition window. Pulse generator module may be coupled to a set of gradient amplifiers to control the timing and shape of the gradient pulses to be produced during the scan. Pulse generator module also receives patient data from a physiological acquisition controller that receives signals from sensors attached to the patient, such as ECG (electrocardiogram) signals from electrodes or respiratory signals from a bellows. Pulse generator module may be coupled to a scan room interface circuit which receives signals from various sensors associated with the condition of the patient and the magnet system. A patient positioning system may receive commands through the scan room interface circuit to move the patient to the desired position for the scan.

The controller 701 may comprise a transceiver module which is configured to produce pulses which are amplified by an RF amplifier and coupled to RF coil by a transmit/receive switch. The resulting signals radiated by the excited nuclei in the patient may be sensed by the same RF coil and coupled through transmit/receive switch to a preamplifier. The amplified nuclear magnetic resonance (NMR) signals are demodulated, filtered, and digitized in the receiver section of transceiver. Transmit/receive switch is controlled by a signal from pulse generator module to electrically couple RF amplifier to coil for the transmit mode and to preamplifier for the receive mode. Transmit/receive switch may also enable a separate RF coil (for example, a head coil or surface coil, not shown) to be used in either the transmit mode or receive mode.

The NMR signals picked up by RF coil may be digitized by the transceiver module and transferred to a memory module coupled to the controller. The receiver in the transceiver module may preserve the phase of the acquired NMR signals in addition to signal magnitude. The down converted NMR signal is applied to an analog-to-digital (A/D) converter (not shown) which samples and digitizes the analog NMR signal. The samples may be applied to a digital detector and signal processor which produces in-phase (I) values and quadrature (Q) values corresponding to the received NMR signal. The resulting stream of digitized I and Q values of the received NMR signal may then be employed to reconstruct an image. The provided methods herein may take the reconstructed image as input and process for MR imaging enhancement and anomaly detection module purpose.

The controller 701 may comprise or be coupled to an operator console (not shown) which can include input devices (e.g., keyboard) and control panel and a display. For example, the controller may have input/output (I/O) ports connected to an I/O device such as a display, keyboard and printer. In some cases, the operator console may communicate through the network with the computer system 710 that enables an operator to control the production and display of images on a screen of display.

The system 700 may comprise a user interface. The user interface may be configured to receive user input and output information to a user. The user input may be related to control of image acquisition. The user input may be related to the operation of the MRI system (e.g., certain threshold settings for controlling program execution, parameters for controlling the joint estimation of coil sensitivity and image reconstruction, etc). The user input may be related to various operations or settings about the MR imaging enhancement and anomaly detection system 740. The user input may include, for example, a selection of a target structure or ROI, training parameters, displaying settings of a reconstructed image, customizable display preferences, selection of an acquisition scheme, and various others. The user interface may include a screen such as a touch screen and any other user interactive external device such as handheld controller, mouse, joystick, keyboard, trackball, touchpad, button, verbal commands, gesture-recognition, attitude sensor, thermal sensor, touch-capacitive sensors, foot switch, or any other device.

The MRI platform 700 may comprise computer systems 710 and database systems 720, which may interact with the controller. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), and/or one or more Advanced RISC Machine (ARM) processors. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

The imaging platform 700 may comprise one or more databases. The one or more databases 720 may utilize any suitable database techniques. For instance, structured query language (SQL) or “NoSQL” database may be utilized for storing image data, raw collected data, reconstructed image data, training datasets, validation dataset, trained model (e.g., hyper parameters), weighting coefficients, etc. Some of the databases may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present disclosure is implemented as a data-structure, the use of the database of the present disclosure may be integrated into another component such as the component of the present disclosure. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

The network 730 may establish connections among the components in the imaging platform and a connection of the imaging system to external systems. The network 730 may comprise any combination of local area and/or wide area networks using both wireless and/or wired communication systems. For example, the network 730 may include the Internet, as well as mobile telephone networks. In one embodiment, the network 730 uses standard communications technologies and/or protocols. Hence, the network 730 may include links using technologies such as Ethernet, 802.11, worldwide interoperability for microwave access (WiMAX), 2G/3G/4G mobile communications protocols, asynchronous transfer mode (ATM), InfiniBand, PCI Express Advanced Switching, etc. Other networking protocols used on the network 230 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), and the like. The data exchanged over the network can be represented using technologies and/or formats including image data in binary form (e.g., Portable Networks Graphics (PNG)), the hypertext markup language (HTML), the extensible markup language (XML), etc. In addition, all or some of links can be encrypted using conventional encryption technologies such as secure sockets layers (SSL), transport layer security (TLS), Internet Protocol security (IPsec), etc. In another embodiment, the entities on the network can use custom and/or dedicated data communications technologies instead of, or in addition to, the ones described above.

Systems and methods of the present disclosure may provide a MR imaging enhancement and anomaly detection system 740 that can be implemented in software, hardware, firmware, embedded hardware, standalone hardware, application specific-hardware, or any combination of these. The MR imaging enhancement and anomaly detection system 740 can be a standalone system that is separate from the MR imaging system. The MR imaging enhancement and anomaly detection system 740 may be in communication with the MR imaging system such as a component of a controller of the MR imaging system. In some embodiments, the MR imaging enhancement and anomaly detection system 740 may comprise multiple components, including but not limited to, a training module, a MR imaging enhancement and anomaly detection module and a user interface module.

The training module may be configured to train the model framework as described above. For instance, the training module may be configured to train a network (e.g., VAE model) for generating anomaly mask and a network (e.g., multi-contrast network) for synthesizing a full-dose MR image (i.e., image enhancement). The training module may train the two models or networks separately. Alternatively or in addition to, the two models may be trained as an integral model.

The training module may be configured to obtain and manage training datasets. For example, the training datasets for the anomaly segmentation or mask generation network (e.g., VAE) may comprise healthy MR image from a subject. The training module may be configured to train the VAE network and multi-contrast network as described elsewhere herein. For example, the VAE network may be trained utilizing the unsupervised anomaly detection (UAD) scheme. The training module may train a model off-line. Alternatively or additionally, the training module may use real-time data as feedback to refine the model for improvement or continual training.

The MR imaging enhancement and anomaly detection module may be configured to perform anomaly mask generation and contrast enhancement using trained models obtained from the training module. The MR imaging enhancement and anomaly detection module may deploy and implement the trained model for making inferences, e.g., predicting UAD mask and synthesizing enhanced MR image.

The user interface module may permit users to view the training result, view predicted results or interact with the training process.

Methods as described herein can be implemented by way of machine (e.g., computer processor) executable code stored on an electronic storage location of the computer system 710, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for enhancing image quality and anomaly detection, the method comprising:
(a) obtaining a multi-contrast image of a subject, wherein the multi-contrast image comprises an image of a first contrast acquired with a reduced dose of contrast agent;
(b) generating an anomaly mask using a first deep learning network; and
(c) taking the multi-contrast image and the anomaly mask as input to a second deep network model to generate a predicted image with improved quality.

2. The computer-implemented method of claim 1, wherein the multi-contrast image is acquired using a magnetic resonance (MR) device.

3. The computer-implemented method of claim 1, wherein the first deep learning network is trained using unsupervised anomaly detection scheme.

4. The computer-implemented method of claim 3, wherein the first deep learning network comprises a variational autoencoder (VAE) model trained only on images without anomaly.

5. The computer-implemented method of claim 1, wherein the multi-contrast image comprises an image of a second contrast that is processed by the first deep learning network for generating the anomaly mask.

6. The computer-implemented method of claim 5, wherein the image of the first contrast is T1-weighted image and the image of the second contrast is selected from the group consisting of T2-weighted image, fluid attenuated inversion recovery (FLAIR), proton density (PD), and diffusion weighted (DWI).

7. The computer-implemented method of claim 1, wherein the second deep network model comprises multiple branches.

8. The computer-implemented method of claim 7, wherein an input to at least one of the multiple branches comprises the image of the first contrast and an image of a different contrast.

9. The computer-implemented method of claim 7, wherein an input to at least one of the multiple branches comprises the image of the first contrast and the anomaly mask generated in (b).

10. The computer-implemented method of claim 7, wherein an input to each of the multiple branches comprises at least the image of the first contrast.

11. The computer-implemented method of claim 7, wherein the predicted image with improved quality is generated based on multiple predictions generated by the multiple branches.

12. The computer-implemented method of claim 1, wherein the anomaly mask is further utilized as an attention mechanism for training the second deep learning network model.

13. The computer-implemented method of claim 1, further comprising displaying the predicted image overlaid with the anomaly mask.

14. A non-transitory computer-readable storage medium including instructions that, when executed by one or more processors, cause the one or more processors to perform operations comprising:
(a) obtaining a multi-contrast image of a subject, wherein the multi-contrast image comprises an image of a first contrast acquired with a reduced dose of contrast agent;
(b) generating an anomaly mask using a first deep learning network; and
(c) taking the multi-contrast image and the anomaly mask as input to a second deep network model to generate a predicted image with improved quality.

15. A computer-implemented method for enhancing image quality and anomaly detection, the method comprising:
(a) obtaining a multi-contrast image of a subject, wherein the multi-contrast image comprises an image of a first contrast acquired with a reduced dose of contrast agent;
(b) providing a deep learning network model comprising a multi-contrast branched architecture; and
(c) taking the multi-contrast image and an anomaly mask as input to the deep network model to generate a predicted image with improved quality.

16. The computer-implemented method of claim 15, wherein the multi-contrast branched architecture comprises a first branch configured to process the image of the first contrast and an image of a second contrast.

17. The computer-implemented method of claim 16, wherein the image of the first contrast is T1-weighted image and the image of the second contrast is selected from the group consisting of T2-weighted image, fluid attenuated inversion recovery (FLAIR), proton density (PD), and diffusion weighted (DWI).

18. The computer-implemented method of claim 16, wherein the multi-contrast branched architecture comprises a second branch to process the image of the first contrast and the anomaly mask.

19. The computer-implemented method of claim 15, wherein the multi-contrast branched architecture comprises at least three branches.

20. The computer-implemented method of claim 19, wherein the predicted image with improved quality is generated based on multiple predictions generated by the at least three branches.
